(19) United States
(12) Patent Application Publication
(10) Pub. No.: US 2021/0031434 A1
(43) Pub. Date: Feb. 4, 2021

(54) A Microfluidic Device for Patterning Cellular Material in a 3D Extracellular Environment

(71) Applicant: Cellink AB, Göteborg (SE)

(72) Inventors: Hector Martinez, Göteborg (SE); Erik Gatenholm, Göteborg (SE)

(21) Appl. No.: 17/046,154

(22) PCT Filed: Apr. 8, 2019
(86) PCT No.: PCT/EP2019/058799

§ 371 Date: Oct. 8, 2020

(30) Foreign Application Priority Data
Apr. 11, 2018 (SE) 1850409-2

(51) Int. Cl. B29C 64/106; C12N 5/00; B01L 3/00; C12M 3/06; B29C 64/209

(52) U.S. Cl. CPC B29C 64/106; C12N 5/0062; C12N 2513/00; C12M 23/16; B29C 64/209; B01L 3/502761

(57) ABSTRACT

The present disclosure relates to a method for arranging cellular material in a bioink, gel or hydrogel material used in 3D-bioprinting. The method comprises bioprinting or dispensing at least one layer of bioink/gel/hydrogel, dispensing or patterning cellular material in the form of single cells, spheroids or cell suspension on or in the bioink/gel/hydrogel layer using a microfluidic device, and repeating previous steps in order to create a 3D tissue model with multiple cell layers. The present disclosure also relates to corresponding microfluidic devices, computer programs and 3D bioprinters.

### TECHNICAL FIELD

The present invention relates to the field 3D bioprinting, and especially to microfluidic devices for transporting cellular material, such as cells and spheroids, to and from a 3D extracellular environment, such as hydrogel or bioink, for use in bioprinting.

### BACKGROUND

The field of 3D printing, and more recently 3D bioprinting (printing of biological material), has emerged over the last decades. Various systems and methods exist for 3D bioprinting of biological material for the purpose of obtaining a printed material in the form of e.g. a construct, a transplant, an organ, a tissue or a scaffold. For many bioprinting applications it is crucial that the bioprinted material is biocompatible with the human or animal body, and hence contamination of the material must be avoided as much as possible. Also, it is a typical requirement that small volumes of fluid containing biological material can be handled, and therefore the features of any devices for handling the biological material are important to optimise.

Typically, the material that is bioprinted is referred to as bioink. The constituents of the bioink may vary depending on application and requirements, but for many applications hydrogel material is used.

Recently, it has become possible to include cellular material in the bioink material, before printing it to the desired bioprinted material, i.e. the construct, tissue, organ, scaffold, transplant or the like. Especially, it is of interest to include cells and/or spheroids to provide the bioprinted material with properties so that the cellular material may grow under suitable conditions.

Non-uniform distribution of the included cellular material is however challenging to obtain. The sensitivity of the cellular material, which requires clean and gentle handling, makes mixing cellular material and bioink material on the fly during printing very challenging. Other approaches of 3D bioprinting by switching back and forth between a predetermined set of mixes of cellular and bioink material may in some circumstances take too long to complete and/or offer too limiting ranges of cellular material in the bioink material.

### SUMMARY OF THE INVENTION

Thus, there remains a need in the art of 3D bioprinting, where 3D extracellular environments such as gels and bioinks are used that include cellular material, to provide technology that allows efficient, clean and gentle handling of the cellular material in order to optimise the outcome of the 3D bioprinting process, and for the viability and growth of the cellular material as constituents of the bioprinted matter. In particular, there remains a need in the art of 3D bioprinting for 3D bioprinting of 3D extracellular environments such as gels and bioinks including a non-uniform distribution of cellular material.

The inventors of the present disclosure have realized that the distribution of cellular material in the 3D extracellular environments can be performed during and/or after the application of the gel or bioink, with or without cellular material already included, by dispensing or removing cellular material via a microfluidic device into or onto the gel or bioink.

Accordingly, the present disclosure relates to a method for placing cellular material in a bioink, gel or hydrogel material before, during and/or after 3D bioprinting. The method comprises bioprinting or dispensing at least one layer of bioink/gel/hydrogel. Notably, this layer may be bioprinted as a first layer of a new construct, or on a previously printed construct, tissue, or other suitable structure.

The method further comprises dispensing, patterning or withdrawing cellular material in the form of single cells, spheroids or cell suspension on or in the bioink/gel/hydrogel layer using a microfluidic device, wherein the cellular material is dispensed or withdrawn in a predefined pattern programmed by a computer. The method may also comprise repeating the above mentioned steps in order to create a 3D tissue model with multiple cell layers.

Further, the method may be initiated by first dispensing, patterning or withdrawing cellular material as described above, and thereafter bioprinting or dispensing at least one layer of bioink/gel/hydrogel. If the method is initiated by dispensing or patterning cellular material using the microfluidic device, such dispensing may be performed in or on a previously printed construct or as a first step in printing a new 3D construct. If the method is initiated by withdrawal of cellular material using the microfluidic device, such dispensing may be performed in or on a previously printed construct.

The present further relates to a microfluidic device for use in dispensing, patterning or withdrawal of cellular material in the form of single cells, spheroids or cell suspension in or on a bioink/hydrogel/gel material for 3D bioprinting purposes. The microfluidic device comprises at least one inlet channel and at least one outlet channel. The microfluidic device further comprises at least one opening for dispensing and/or withdrawing material. Such openings or outlets may be in the form of one or more nozzles for dispensing and/or withdrawing material. The microfluidic device also comprises at least one storage chamber for storing the cellular material, which storage chamber may be integrated with the device or in communication with the device. The microfluidic device comprises at least one pump in communication with the at least one inlet channels and/or the at least one outlet channels, and/or at least one pressure source for controlling the flow of cellular material in and from the microfluidic device. The microfluidic device also comprises a controller programmed to control the flow of material, within the microfluidic device, and/or into and out from the microfluidic device.

In one aspect, the microfluidic device may comprise one or more sensors in communication with the controller, wherein the one or more sensors are configured to provide data relating to the properties of the cellular material and/or the surrounding environment.

The present disclosure also relates to a computer program for use in dispensing, patterning or withdrawal of cellular material in the form of single cells, spheroids or cell suspension in or on a bioink/hydrogel/gel material for 3D bioprinting purposes. The computer program comprises computer program code which, when executed, causes a processor to carry out the method as disclosed above and below.

The present disclosure further relates to a system for use in dispensing/patterning of cellular material in the form of single cells, spheroids or cell suspension in or on a bioink/hydrogel/gel material for 3D bioprinting purposes. The system comprises a microfluidic device as described above and below. The microfluidic device further comprises a least one nozzle or outlet for dispensing bioink and/or hydrogel. The system also comprises control circuitry configured to carry out the method as described above and below.

The disclosed method, microfluidic device, computer program and system all enable the technical effect of providing 3D extracellular environments such as gels and bioinks and bioprinted constructs having a non-uniform distribution of cellular material in a manner that includes efficient, clean and gentle handling of the cellular material. Further, an efficient and exact placement of desired cellular material is obtained within or on a bioprinted construct.

Notably, the term “non-uniform” encompasses all form of distribution of cellular material where the cellular material is intentionally placed in a at least generally predefined pattern. This is in contrast to a uniform (homogenous) distribution that is obtained when a cellular material is mixed into the bioink, gel or hydrogel material before performing a 3D-bioprinting procedure, such that the cellular material is evenly spread our through the final construct. Notably, a non-uniform pre-defined pattern may also comprise any type of symmetrical or evenly distributed pattern of cellular material.

The non-uniform distribution of cellular material includes both non-uniform distribution of cellular material of the same type, as well as cellular material of different types. For instance, non-uniform distribution of cellular material may comprise stacking cellular material of different types, e.g. different types of cells and/or spheroids, in a certain order and/or arranging the cellular material of different types in a pattern, e.g. a two-dimensional pattern within one or more 3D-printed layers, based on the different types of cellular material, or a three-dimensional pattern within one or more 3D-printed layers, where the cellular material may be of the same type throughout, or of different types. If using a microfluidic device with multiple openings, as will be described further below, cellular material may be placed in one or several layers at multiple points from different openings simultaneously.

In an example of cellular material of different types stacked in different layers, for three types of cells, a first cell type may be arranged in a first printed layer, a second cell type may be arranged in a second printed layer and a third cell type may be arranged in a third printed layer.

In an example of cellular material of different types arranged non-uniformly within a printed layer, for three types of cells, a first region comprising a first cell type may be arranged adjacent to a second region comprising a second cell type. The second region may in turn be arranged adjacent to a third region comprising the first cell type. The third region may further be arranged adjacent to a fourth region comprising a third cell type.

The above examples are for illustrative purposes only, and the non-uniform distribution may comprise any arrangement of alternating printed layers and/or possible patterns within and/or between printed layers. In other words, the disclosed method, microfluidic device, computer program and system enable 3D-bioprinting of any non-uniform distribution of cellular material of different types.

### BRIEF DESCRIPTION OF THE DRAWINGS

FIG. 1 discloses different microfluidic devices and an exemplary use of such a device;

FIG. 2 discloses a method for 3D-bioprinting a construct;

FIG. 3 discloses a system for 3D-bioprinting a construct; and

FIG. 4 discloses yet another microfluidic device.

### DEFINITIONS

A “microfluidic device” is meant to refer to the microfabricated device comprising microchannels or circuitries of microchannels, which are used to handle and move fluids. Preferably, microfluidic devices may include components like junctions, reservoirs, valves, pumps, mixers, filters, chromatographic columns, electrodes, waveguides, sensors etc.

Microfluidic devices can be made of polymers such as polydimethylsiloxane (PDMS), poly (methyl methylacrylate) (PMMA), polytetrafluoroethylene (PTFE), polyethylene (PE) epoxy resins, and thermosetting polymers; amorphous materials (e.g., glass), crystalline materials (e.g., silicon, silicon dioxide); or metallic materials (e.g., aluminum, copper, gold, and silver, and alloys thereof). In certain preferred embodiments, a microfluidic device may contain composite materials or may be a composite material. The microfluidic device may be a microfluidic pipette or pen, or part of or connected to a printhead for a bioprinter.

“Cellulose nanofibril based bioink” refers to a dispersion of cellulose nanofibrils in a liquid media (see WO2016/100856 for further definitions). E.g. the cellulose nanofibrils have a length of about 1-100 microns and a width of about 10-30 nanometers; a viscosity of between 0.01 and 100 Pa's at 100 s^{″1}; a solids content ranging from about 0.1-40%.

“Bioprinting” refers to the utilization of 3D printing and 3D printing-like techniques to combine cells, growth factors, and biomaterials to fabricate biomedical parts that maximally imitate natural tissue characteristics. Generally, 3D bioprinting utilizes the layer-by-layer method to deposit materials known as bioinks to create tissue-like structures that are later used in medical and tissue engineering fields.

By “physiological conditions” are meant that that the culture or the cells are exposed to conditions (such as pH, osmolarity, temperature and printing pressure (which is equal to extrusion pressure in this context) that are typical to the normal environment for the culture or cells, such as, for human cells, a temperature around 37° C., such as in the interval from 35-39° C., a printing pressure in the interval from 1 kPa to 200 kPa, preferably below 10 kPa, a pH in the interval from 5-8, preferably about 7, and an osmolarity in the interval from 275 to 300 mOsm/kg, preferably about 295 mOsm/kg.

Spheroid is a set of cells confined within a spherical space having a predetermined diameter range, e.g. between 30 μm and 300 μm, preferably below 100 μm. The set of cells may be lumped together and/or embedded within an essentially spherical extracellular environment such as a gel or a bioink, whereon the spherical extracellular environment has a diameter within the predetermined range.

### DETAILED DESCRIPTION OF THE INVENTION

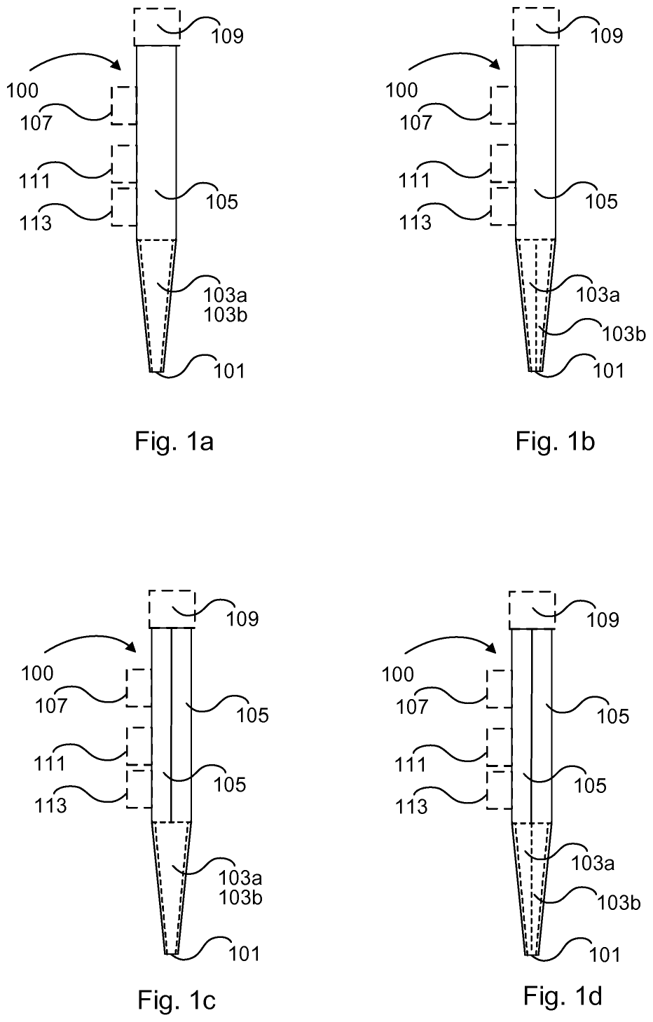

FIG. 1a-1d disclose four different exemplary microfluidic devices 100, as will be described further below.

FIG. 1e discloses a schematic of using a microfluidic device 100 according to any of the herein disclosed types, for transporting cells (small circles) 102a, 102b and/or spheroids (larger circles including several cells) 104a, 104b to or from a 3D extracellular environment 106, such as a bioink or gel construct used in bioprinting.

The result of using the microfluidic device 100 is a 3D-bioprinted construct in the form an implant, an organ, a tissue, tissue model/analogue, or a scaffold, wherein the 3D-bioprinted construct is formed by the use of the microfluidic device.

The Microfluidic Device

The microfluidic device 100 of the present invention may comprise one or more microfluidic outlet channels 103b and one or more microfluidic inlet channels 103a. In FIGS. 1a and 1c the channels are illustrated as being a single channel adapted to be both an inlet and an outlet channel. As an alternative, the inlet and outlet channels may be configured as two or more separate channels, as seen in FIGS. 1b and 1d, wherein the channels may or may not be identical in shape or configuration. The one or more outlet channels 103b and the one or more inlet channels 103a comprise at least one opening 101, so that substance dispensed from the device 100 to a surrounding material, such as a bioink or a hydrogel, via the one or more outlet channels passes the at least one opening, and so that substance withdrawn from the surrounding material, such as a bioink or a hydrogel, via the one or more inlet channels passes the at least one opening. It is also conceivable that each channel have a separate opening.

The microfluidic device comprises at least one pump in communication with the at least one inlet channels and/or the at least one outlet channels, and/or at least one pressure source for controlling the flow of cellular material in and from the microfluidic device. The microfluidic device 100 may comprise a low pressure electronic regulator 107 as a means to control the pressure applied to the cells/cellular material in suspension. According to some aspects, this low pressure regulator can control the pressure within a range of 10 in H₂O (2488 Pa) and at an accuracy of up to ±0.02 in H₂O (5 Pa).

The microfluidic device 100 comprises a storage chamber 105 for storing the cellular material, e.g. in the form of a cartridge. Such a storage chamber 105 may be integrated into the microfluidic device, or provided as a separate and connectable cartridge. According to some aspects, the storage chamber has a volume of 0.1, 1, 2, 3, 5, 10, 30, 50 ml.

At one end, the storage chamber 105 may be connected to a pressure input that is controlled by the low pressure electronic regulator to control the flow rate of the cellular material. According to some aspects, a micro-filtered, oil-free air pressure source is used as the input for the pressure regulator. The inlet and outlet channels of the microfluidic device can be embedded directly inside a nozzle that is attached to the cartridge. Alternatively, the inlet and outlet channels of the microfluidic device can be connected to the cartridge by using a small tubing or mounted directly to the cartridge though a luer lock connection. As an example, a custom-made microfluidic device can be manufactured utilizing two-photon induced polymerization technology to create a tip with microchannels and a luer lock connection for easy attachment to a storage cartridge.

In another example, the cellular material can be stored directly in the microfluidic device, containing a storage chamber 105, one or more inlets to the storage chamber (inlets for air and cell material can be combined or separate), microchannels 103a,103b and/or one or more openings. Such a custom-made microfluidic device can be manufactured utilizing two-photon induced polymerization technology.

In some embodiments, such as seen in FIGS. 1a-1d, the one or more outlet channels and one or more inlet channels are identical in shape, either as two separate channels (FIGS. 1b and 1d), or one and the same channel (FIGS. 1a and 1c). The one or more channels may function both as outlet and inlet channels.

Thus, in some embodiments the one or more outlet channels 103b and one or more inlet channels 103a are separate, as illustrated in FIGS. 1b and 1d. Further, the microfluidic device 100 may comprise two or more storage chambers 105, as shown in FIGS. 1c and 1d. Such storage chambers 105 may be arranged to either deliver, receive or both deliver and receive material from the channels. In some embodiments the one or more outlet channels and the one or more inlet channels are in communication with each other and/or the one or more storage chambers via a common channel or reservoir (not seen in the Figures). Thus the microfluidic device may be adapted to suit any need for dispensing material, withdrawing material and/or mixing of material.

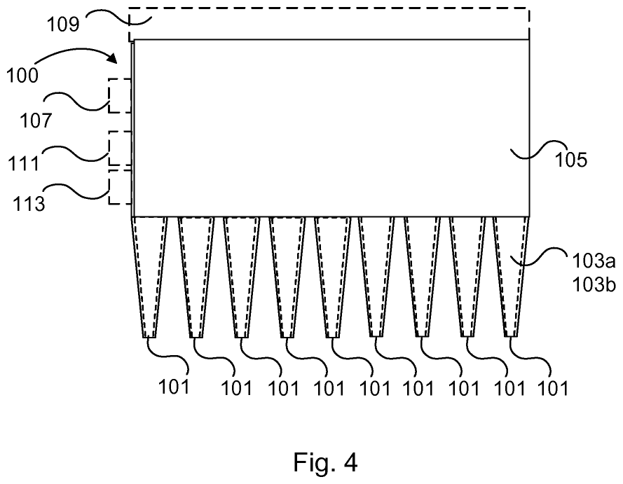

In one aspect, as illustrated in FIG. 4, the microfluidic device 100 may be provided with multiple sets of inlet and/or outlet channels 103a, 103b, connected or connectable to one or more a common storage chambers 105. Thus, in such a microfluidic device, the channels may be provided in multiple nozzles, arranged such that the openings 101 form a predetermined pattern. One example is shown in FIG. 4, where nozzles with openings 101 are arranged in a single line. However, any arrangement and number of openings is conceivable, such as any two- or three-dimensional pattern of openings. An example could be a set of nozzles arranged with openings in an array of a specified number of rows and columns in an array, such as 2×2, 2×4, 4×4, 4×6, 6×6, 6×8, 8×8 or any desired arrangement. Further the openings may be arranged in the same plane or in different planes, effectively forming a three-dimensional pattern.

In FIG. 4, the microfluidic device 100 is shown with common inlet and outlet channels and one single storage chamber 105. However, in other aspects such a microfluidic device may be provided with separate inlet and outlet channels, in each nozzle or in separate nozzles. The microfluidic device may further be provided with two or more storage chambers, as described in connection to FIGS. 1a-1d. When using multiple storage chambers, each storage chamber may be connected to one or more channels, and/or one or more nozzles in the device, such that any combination of dispensing, withdrawal and/or patterning may be provided.

The one or more outlet channels and the one or more inlet channels may have a total cross-sectional smallest dimension so that individual cells and/or spheroids can be dispensed from or withdrawn to the microfluidic device in a controllable manner. For example, the cross-sectional smallest dimension may be in the interval from 1 to 100 micrometers, for example about 1, 5, 10, 15, 20, 25, 30, 35, 40, 45, 50, 55, 60, 65, 70, 75, 80, 85, 90, 95, or 100 micrometers (μm). The cross-sectional smallest dimension may exceed 100 μm. A smallest cross-sectional dimension of 100 μm or larger is suitable when printing adult human pancreatic islets, which measure an average of 100 μm in diameter. Other applications include printing of spheroids which are much larger, e.g. pancreatic islets from mouse. Thus, the cross-sectional smallest dimension may exceed 400 μm.

The one or more outlet and inlet channels may have a length of about 0.5 to 30 cm, for example about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 cm.

Further, the microfluidic device may comprise a first pump 109 in communication with the one or more outlet channels and/or a second pump 109 in communication with the one or more inlet channels.

Moreover, the microfluidic device comprises a controller 111 programmed to control the flow of fluid material within the microfluidic device, and/or into or out from the microfluidic device. Thus, the controller may control the flow of material within one or more microfluidic outlet channels and microfluidic inlet channel(s). The controller may also control the flow of material out from and/or into the channels through the opening. Such a controller 111 enables improved control of handling of the cellular material, adding to the desired efficient, clean and gentle handling of such material, which results in optimised outcome of the 3D bioprinting process and improved viability and growth of the cellular material as constituents of the bioprinted matter. Furthermore, using a controller results in improved efficiently and accuracy in obtaining a desired pattern or distribution of cellular material.

The microfluidic device may comprise one or more sensors 113 in communication with the controller, wherein the one or more sensors are configured to provide data relating to the properties of the stored cellular material and/or the surrounding environment, such as the hydrogel or bioink layer wherein material is to be dispensed or withdrawn. As non-limiting examples, such sensor(s) may be adapted to provide data relating to one or several parameters such as density, water content, temperature, hardness, humidity, pH etc. As one example, such sensors maybe configured to monitor the status of the cellular material in the storage chamber, such as monitoring of temperature and/or pH, to ensure optimal viability of the cellular material. A sensor may also monitor the amount of cellular material in the storage chamber, which allows improved control of knowing when to replace a cartridge or fill up the storage chamber. Further, as another example, sensors may be adapted to provide data on e.g. the thickness of a hydrogel or bioink layer wherein material is to be dispensed or withdrawn, which in turn may be used to determine if dispensing in a desired manner is possible.

The microfluidic device may comprise a positive pressure source in communication with the one or more microfluidic outlet channels, and/or a negative pressure source in communication with the one or more microfluidic inlet channels.

The microfluidic device may be provided as a hand-held device, for example in the form of a pipette or a pen. Further, the microfluidic device may be provided as a print nozzle or printhead for a 3D bioprinter, or as part of or attachable to such a printhead. In addition, or as an alternative, the microfluidic device may be provided as part of or attachable to a system for controlled positioning of the cellular material.

Bioink or (Hydro)Gel

The present invention may be used in relation to any available bioink material or gel, as long as the material provides proper biocompatibility and bioprinting properties.

Nanocellulose Bioink Material

Some embodiments of the present invention relate to biomaterial in liquid or gel form (e.g., dispersions) defined as a bioink which can be used for 3D bioprinting of scaffolds, tissues and organs. More particularly, some embodiments of the invention include the use of the bioink from nanocellulose material with and without cells to bioprint 3D scaffolds, 3D cell culture models, tissues and organs.

Bioink, CELLINK®, as described in this invention is preferably composed of a nanofibrillated cellulose dispersion with preferable addition of a crosslinking component. Such bioink can be crosslinked preferably after printing or even during the 3D bioprinting operation. In some applications CELLINK® can be used without a crosslinking agent. CELLINK® as described in this invention has unique rheological and gelation properties. That is, it exhibits shear-thinning behavior, high zero-shear viscosity, a fast response to re-establish the high zero-shear viscosity after extrusion and a rapid gelation to avoid deformation of the bioprinted construct. These rheological properties make it possible to obtain a high spatial resolution of the bioprinted construct and to maintain a high shape fidelity during the bioprinting process. The viscosity of CELLINK® can be tailor-made by selecting a suitable concentration of cellulose nanofibrils, their length (aspect ratio), charge and additives. Desired cytotoxicity characteristics and cell viability characteristics have been developed by a purification process and adaptation of osmolarity of the dispersion in order to print CELLINK® with living cells.

Embodiments of the invention include cellulose nanofibril bioink products prepared by the methods described and include using the products in 3D Bioprinting operations. Cellulose can be generated from plants (such as annual plants), trees, fungi or bacteria, with preferred embodiments generated from bacteria such as from one or more of the genera Aerobacter, Acetobacter, Acromobacter, Agrobacterium, Alacaligenes, Azotobacter, Pseudomonas, Rhizobium, and/or Sarcina, specifically Gluconacetobacter xylinus, Acetobacter xylinum, Lactobacillus mali, Agrobacterium tumefaciens, Rhizobium leguminosarum bv. trifolii, Sarcina ventriculi, enterobacteriaceae Salmonella spp., Escherichia coli, Klebsiella pneumoniae and several species of cyanobacteria.

Cellulose can be generated from any vascular plant species, which include those within the groups Tracheophyta and Tracheobionta. Cellulose nanofibrils formed from cellulose producing bacteria most closely mimic the characteristics of collagen found in human and animal soft tissue. The array of fibrils provides a porous yet durable and flexible material. The nanofibrils allow nutrients, oxygen, proteins, growth factors and proteoglycans to pass through the space between the fibrils, allowing the scaffold to integrate with the implant and surrounding tissue. The nanofibrils also provide the elasticity and strength needed to replace natural collagen. The bacterial cellulose materials have been, after purification, homogenized and hydrolyzed to smooth dispersion. WO2016/100856 is hereby incorporated as a reference for the cellulose-based bioink material.

Wood-derived cellulose nanofibrils were selected as an alternative raw material for the preparation of cellulose nanofibrillated bioink. The difference is that they do not form a three-dimensional network and their width is lower (10-20 nanometers) and length is lower (1-20 micrometers). The disadvantage of the wood derived cellulose nanofibrils can be the presence of other wood biopolymers such as hemicelluloses which can affect cells and cause foreign body reaction. These dispersions should preferably therefore be purified by an extraction process and removal of the water phase. It is a sensitive process since it can lead to agglomeration of fibrils which can result in bioink which tends to clog the 3D bioprinter printing nozzle. For use in the present disclosure homogenization is used followed by centrifugation and ultrafiltration to prepare bioink based on wood cellulose nanofibrils. It has been found that the optimal properties were achieved when dispersion with solid content above 1% dry matter were used. The nanocellulose-based bioink has been purified by replacing the water with pyrogen-free water by consecutive centrifugation and resuspension steps. Then the osmolarity was adjusted for cells by dissolving of D-mannitol directly in the nanocellulose hydrogel to a final concentration of 4.6% D-mannitol (w/v). Alternatively to using 4.6% D-mannitol, cell culture media can be used to replace the water content in the nanocellulose bioink utilizing the same method of consecutive centrifugation and resuspension steps until all water content is replaced by cell culture media. The sterilization procedure was performed using electron beam, EB, sterilization at 25 kGy. No effect on viscosity or stability of nanocellulose dispersion was observed after the sterilization process. The nanocellulose bioink can be 3D bioprinted without addition of cross-linker or biopolymer acting as binder.

Nutrients, oxygen, proteins, growth factors and proteoglycans can pass and diffuse through the space between the fibrils. Embodiments are designed to allow cells to stay in the bioink and are able to support extracellular matrix production which results in tissue formation without contraction.

Another advantageous characteristic of nanofibrillated bioinks is that they can be non-degradable. Most biologically occurring materials are degradable, meaning they will break down or deteriorate over time, which can be problematic for use as disease models, for drug screening or for soft tissue repair. A non-degradable biological material provides a biologically compatible scaffold that will tend to maintain structure and function, or maintain structure and/or function for a desired period of time (such as the length of anticipated testing). Moreover, materials with good mechanical properties are provided, which properties are desired for use of the constructs as implants.

In another variant of the bioink at least one additional biopolymer is added to the bioink, wherein the biopolymer gelling agent or hydrocolloid is chosen from the group comprising alginates, hyaluronic acid and its derivatives, agarose and its derivatives, chitosan, fibrin, gellan gum, crystalline nanocellulose, carrageenans, collagen and its derivatives as well as gelatin and its derivatives. These additional biopolymers are added to the bioink for crosslinking purposes and/or to contribute to rheological properties as hydrocolloids or thickening agents. Addition of cross-linker or binding biopolymers such as alginate can be used to improve printability but also provide mechanical stability after crosslinking with 100 mM buffered calcium chloride solution or lower concentrations of the same crosslinking agent. In another variant of the bioink, the nanocellulose bioink is used as support material for printing of pH neutralized collagen solution in combination with extracellular matrix. The nanocellulose component will help keep the 3D shape of the printed construct due to its shear-thinning behavior and high zero-shear viscosity. This allows for printing of a complex 3D support, which can, after gelation of collagen solution and/or extracellular matrix, be removed.

One advantage of using cellulose nanofibril hydrogels is the extreme shear thinning properties which is crucial for high printing fidelity. High printing fidelity makes it possible to bioprint porous structures which can be spontaneously vascularized upon implantation.

Vascularization is a key factor to promote engraftment with the host tissue, since vascularization makes it possible for oxygen and nutrient transport throughout the bioprinted construct. In addition, the cells can migrate through porosity to enhance tissue formation. Another advantage of using cellulose nanofibrils is their large surface area and hydrophilic properties which make them an excellent binder and dispersing agent for organic and inorganic particles.

Alternative Bioink Materials

Further examples of suitable bioink materials for use in the present invention to be combined with ECM material, with or without cells, are chosen, but not limited to, bioinks comprising at least one of the following constituents: cellulose derivatives, such as cellulose nanofibrils, hyaluronic acid, alginate, agar, pectin, chitosan, gellan gum and carrageenan, or a combination of these constituents, or alternatively a non-polysaccharide based bioink such as collagen or gelatin-based, combined with a polysaccharide-based thickening agent. For example, the following list of available bioinks can be used: CELLINK RGD, CELLINK BONE, CELLINK A, CELLINK A-RGD, CELLINK Collagen (in solution and fibrillary form), CELLINK CollMaGel, CELLINK GelMa, and chitosan-based bioinks.

See also www.cellink.com for further information on the bioinks listed above. This link is included as a reference in the present application.

Especially when using alternative bioink materials, it may be preferable to add a thickening agent (as disclosed in this application), e.g. in the form of a nanocellulose hydrogel (chosen from nanofibrillated cellulose, microfibrillated cellulose, crystalline nanocellulose and bacterial nanocellulose) to the polysaccharide-based bioink, in order to create an optimal microenvironment for the 3D-bioprinted material. Also, a polysaccharide-based thickening agent may also be combined with a collagen or gelatin-based bioink (i.e. a polypeptide-based bioink).

Gel/Hydrogel

Examples of gels/hydrogels comprise MatriGel®, Cultrex®, Collagen (preferably type I, but not limited to this), Hyaluronic acid, fibrinogen with or without thrombin, laminins.

### Example 1

In a first example, the disclosure refers to a microfluidic device in the form of a pipette or a pen that can release cells and/or spheroids in a 3D extra cellular environment. When using the microfluidic pen, cells and/or spheroids can be dispensed from the microfluidic pen into a bioink or gel before, during and/or after bioprinting. Such a microfluidic pen may also be used to withdraw cells and/or spheroids from a construct in a predetermined pattern.

### Example 2

In a second example, the disclosure refers to a microfluidic device in the form of a pipette or a pen that can pick up cells and/or spheroids from a solution or petri dish or the like, and then dispense them in a bioink or gel before, during and/or after bioprinting.

### Example 3

In a third example, the disclosure refers to a microfluidic device in the form of a print nozzle or printhead in a 3D bioprinter device. The print nozzle may be made out of a wide variety of diameters as exemplified above, e.g. 10 μm to 500 μm, so that the size of its output can be adjusted, and so that it therefore can print individual cells as well as spheroids. Cellular material may be dispensed inside an already printed 3D hydrogel or bioink construct in any desired pattern.

### Example 4

In a fourth example, the disclosure refers to a microfluidic device in the form of a print nozzle or printhead in a 3D bioprinter device. The print nozzle mat be adapted to pick up cells and/or spheroids from a solution or petri dish or the like, and then dispense them in a bioink or gel before, during and/or after bioprinting. The print nozzle may further be adapted to withdraw material from a 3D printed construct in a specific pattern.

Method of Arranging Cellular Material in a 3D Construct

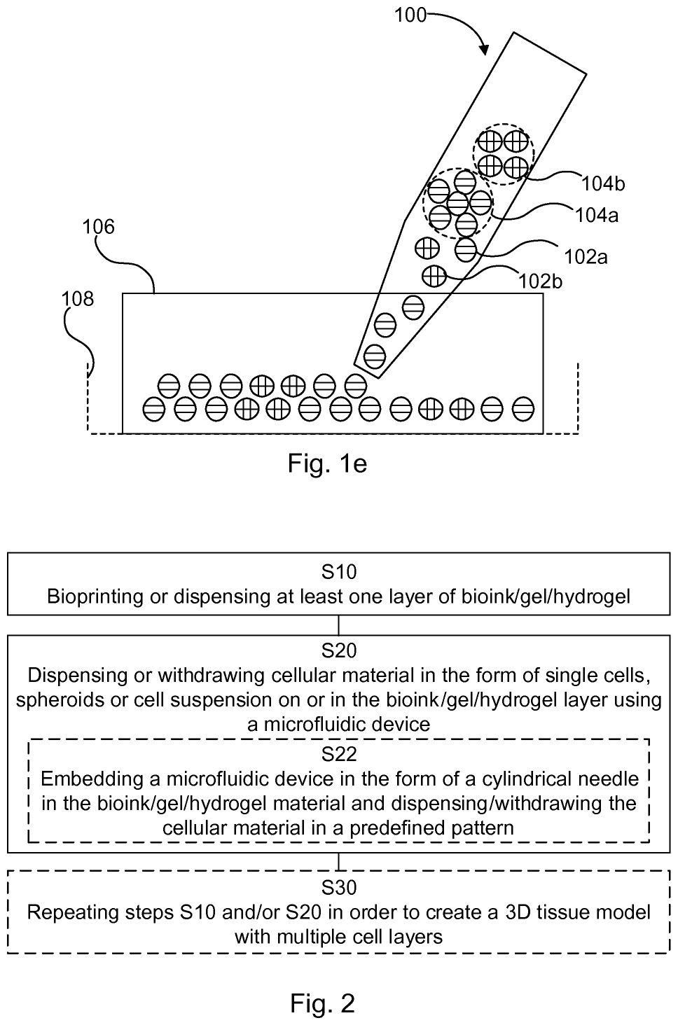

FIG. 2 discloses a method for arranging cellular material in a bioink, gel or hydrogel material before, during and/or after 3D-bioprinting. The term “arranging cellular material” may encompass placing material in or on and/or withdrawing material from e.g. a 3D printed construct. The method comprises bioprinting or dispensing S10 at least one layer of bioink/gel/hydrogel. The method further comprises dispensing, patterning or withdrawing S20 cellular material in the form of single cells, spheroids or cell suspension on or in the bioink/gel/hydrogel layer using a microfluidic device. The method may also comprises repeating S30 one of or both steps S10 and S20 in order to create a 3D tissue model with multiple cell layers. The cellular material is dispensed and/or withdrawn in a predefined pattern programmed by a computer.

The steps of bioprinting or dispensing S10 at least one layer of bioink/gel/hydrogel, and dispensing, patterning or withdrawing S20 cellular material may be performed in any desired order or number of times. As one example, a layer of bioink/gel/hydrogel is bioprinted on a print surface, or other construct, thereafter cellular material is dispensed in a predefined pattern on or in the first layer. As another example, cellular material is first dispensed in a predefined pattern on a print surface, or on or in a layer of bioink/gel/hydrogel and thereafter part of the cellular material is withdrawn in another predefined pattern in the at least one layer of bioink/gel/hydrogel. As yet another example, cellular material is first dispensed, either directly on a print surface, or on a layer of bioink/gel/hydrogel, and thereafter another layer of bioink/gel/hydrogel is bioprinted on top of the dispensed cellular material.

According to some aspects, each bioink/gel/hydrogel layer has a thickness in the interval from 10-400 micrometers. According to further aspects, the cellular material is dispensed/patterned and/or withdrawn directly on each bioink/gel/hydrogel layer.

According to some aspects, the bioink/gel/hydrogel layer has a thickness below 100 micrometers when dispensing/patterning single cells or cell suspension, and a thickness above 100 micrometers when dispensing/patterning cell spheroids.

According to some aspects, the cellular material is dispensed, patterned, and/or withdrawn by embedding S22 a microfluidic device in the form of a needle or nozzle in the bioink/gel/hydrogel material. Such a needle or nozzle is preferably cylindrical, but may also be of any desired cross-sectional shape, such as an elliptical, square, rectangular, triangular, pentagonal, or hexagonal shape etc. The nozzle may also comprise a blunt, tapered or slanted tip. The microfluidic device may be any of the microfluidic devices disclosed herein. An exemplary use of the method using a microfluidic device as disclosed herein is schematically illustrated in FIG. 1e.

According to some aspects, the microfluidic device is pneumatic-driven, and wherein the cellular material, before dispensing, is resuspended in cell culture media and thereafter loaded in a cartridge that is connectable to the microfluidic device.

According to some aspects, the method is adapted for dispensing, patterning and/or withdrawing cellular material in the form of single cells, spheroids or cell suspension larger than about 150 micrometer in diameter.

According to some aspects, the microfluidic device is inkjet-based and wherein the dispensing is performed with an electromagnetic valve.

According to some aspects, the microfluidic device is (a) in the form of a hand-held device, such as a pen or a pipette, (b) in the form of a print nozzle, or (c) attached to a system for controlled positioning of the cellular material.

The present disclosure also relates to computer programs comprising computer program code which, when executed, carries out the method as disclosed above and below.

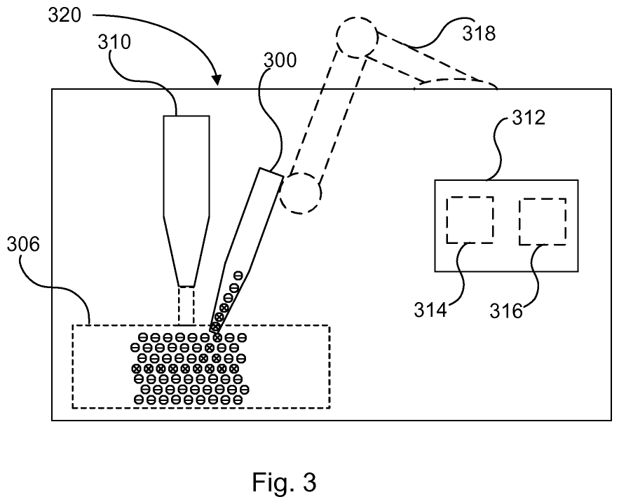

FIG. 3 discloses a system 320 for 3D-bioprinting a construct 306 in the form of an implant, an organ, a tissue, tissue model/analogue, or a scaffold. The system 320 comprises a microfluidic device 300 for transporting cells and/or spheroids to and/or from a hydrogel and/or a bioink material, as described above and below. The system 320 further comprises a nozzle 310 for dispensing bioink and/or hydrogel, such as a bioprinting nozzle. The system 320 also comprises control circuitry 312 configured to carry out the method for 3D-bioprinting a construct in the form of an implant, an organ, a tissue, tissue model/analogue, or a scaffold, as described above and below.

According to some aspects, the control circuitry comprises a processor 314 and a memory 316. The memory 316 is configured to store a computer program as described above and below thereon. The processor 314 is configured to execute the computer program when stored on the memory 316.

According to some aspects, the system further comprises a robotic arm 318, wherein the robotic arm 318 is configured to position the microfluidic device with respect to the construct when 3D-bioprinting the construct. According to some yet further aspects, the robotic arm is configured to move in six degrees of freedom (three translational and three rotational).

The present invention is not limited to the above-described preferred embodiments. Various alternatives, modifications and equivalents may be used. Therefore, the above embodiments should not be taken as limiting the scope of the invention, which is defined by the appending claims.

1. A method for arranging cellular material in a bioink, gel or hydrogel material before, during and/or after 3D-bioprinting, comprising the steps of:
(a) bioprinting or dispensing at least one layer of bioink/gel/hydrogel;
(b) dispensing or patterning or withdrawing cellular material in the form of single cells, spheroids or cell suspension on or in a bioink/gel/hydrogel layer using a microfluidic device, wherein the cellular material is dispensed, patterned or withdrawn in a predefined pattern programmed by a computer, step b being performed before or after step a;
(c) optionally repeating one of or both steps a. and b. in order to create a 3D tissue model with multiple cell layers.

2. The method according to claim 1, wherein cellular material is first dispensed in a predefined pattern and thereafter withdrawn in another predefined pattern in at least one layer of bioink/gel/hydrogel.

3. The method according to claim 1, or wherein each bioink/gel/hydrogel layer has a thickness in the interval from 10-400 micrometers.

4. The method according to claim 1, wherein the cellular material is dispensed/patterned and/or withdrawn directly on each bioink/gel/hydrogel layer.

5. The method according to claim 3, wherein the bioink/gel/hydrogel layer has a thickness below 100 micrometers when dispensing/patterning single cells or cell suspension, and a thickness above 100 micrometers when dispensing/patterning cell spheroids.

6. The method according to claim 1, wherein the cellular material is dispensed or withdrawn by embedding the tip of a needle or nozzle of a microfluidic device in the bioink/gel/hydrogel material.

7. The method according to claim 1, wherein the microfluidic device is pneumatic-driven, and wherein the cellular material, before dispensing, is resuspended in cell culture media and thereafter loaded in a cartridge that is connectable to the microfluidic device.

8. The method according to claim 1, for dispensing and/or withdrawing cellular material in the form of single cells, spheroids or cell suspension larger than about 150 micrometer in diameter.

9. The method according to claim 1, wherein the microfluidic device is inkjet-based and wherein the dispensing or withdrawing of material is performed with an electromagnetic valve.

10. The method according to claim 1, wherein the microfluidic device is (a) in the form of a hand-held device, or (b) in the form of a print nozzle, and/or (c) attached to a system for controlled positioning of the cellular material.

11. A microfluidic device for use in dispensing or withdrawal of cellular material in the form of single cells, spheroids or cell suspension in or on a bioink/hydrogel/gel material for 3D bioprinting purposes, wherein the microfluidic device comprises:
at least one outlet channel and inlet channel;
at least one opening for dispensing and/or withdrawing material;
at least one storage chamber for storing the cellular material, which storage chamber may be integrated with the device or in communication with the device;
at least one pump in communication with the at least one inlet channels and/or the at least one outlet channels; and/or
at least one pressure source for controlling the flow of cellular material within, and/or into and out from the microfluidic device; and
a controller programmed to control the flow of material.

12. The microfluidic device according to claim 11, further comprising one or more sensors in communication with the controller, wherein the one or more sensors are configured to provide data relating to the properties of the cellular material and/or the surrounding environment.

13. The microfluidic device according to claim 11, wherein said outlet and inlet channels are configured as a common channel, adapted to function both as outlet and inlet channel.

14. The microfluidic device according to claim 11, wherein said outlet and inlet channels are configured as separate outlet and inlet channels.

15. The microfluidic device according to claim 11, wherein the storage chamber is in the form of a cartridge that is connectable to the microfluidic device.

16. The microfluidic device according to claim 11, wherein the microfluidic device is provided as a hand-held device, such as a pen or a pipette.

17. The microfluidic device according to claim 11, wherein the microfluidic device is provided as a print nozzle or printhead for a 3D bioprinter.

18. The microfluidic device according to claim 11, wherein the microfluidic device is provided as part of or attachable to a system for controlled positioning of the cellular material.

19. The microfluidic device according to claim 11, wherein the device is pneumatic-driven or inkjet-based.

20. The microfluidic device according to claim 11, wherein the outlet and/or inlet channels have a length of about 0.5-30 cm, and a cross-sectional smallest dimension in the interval of 1-1000 μm.

21. A computer program for use in dispensing and/or withdrawal of cellular material in the form of single cells, spheroids or cell suspension in or on a bioink/hydrogel/gel material for 3D bioprinting purposes, the computer program comprising computer program code which, when executed, causes a processor to carry out the method according to claim 1.

22. A system for use in dispensing and/or withdrawal of cellular material in the form of single cells, spheroids or cell suspension in or on a bioink/hydrogel/gel material for 3D bioprinting purposes, the system comprising
a microfluidic device according to claim 11,
a bioprinting nozzle for dispensing bioink and/or hydrogel, and control circuitry configured to carry out a method for arranging cellular material in a bioink, gel or hydrogel material before, during and/or after 3D-bioprinting, wherein the method comprises:
(a) bioprinting or dispensing at least one layer of bioink/gel/hydrogel; and
(b) dispensing or patterning or withdrawing cellular material in the form of single cells, spheroids or cell suspension on or in a bioink/gel/hydrogel layer using a microfluidic device, wherein the cellular material is dispensed, patterned or withdrawn in a predefined pattern programmed by a computer, (b) being performed before or after (a); and
optionally repeating one of or both (a) and (b) in order to create a 3D tissue model with multiple cell layers.

23. The system according to claim 22, wherein the control circuitry comprises a processor and a memory, wherein the memory is configured to store a computer program thereon, wherein the computer program is a computer program for use in dispensing and/or withdrawal of cellular material in the form of single cells, spheroids or cell suspension in or on a bioink/hydrogel/gel material for 3D bioprinting purposes, the computer program comprising computer program code which, when executed, causes a processor to carry out the method according to (a) and (b), and wherein the processor is configured to execute the computer program when stored on the memory.

24. The system according to claim 22, further comprising
a robotic arm,
wherein the robotic arm is configured to position the microfluidic device with respect to the construct before, during and/or after 3D-bioprinting the construct.

25. A 3D-bioprinted construct in the form of an implant, an organ, a tissue model, a scaffold, wherein the 3D-bioprinted construct is formed by employing the method of claim 1, and wherein the construct has a pre-determined distribution of cellular material in the form of single cells, spheroids or cell suspension on or in at least one bioink/gel/hydrogel layer.
